(19) United States
(12) Patent Application Publication
(10) Pub. No.: US 2020/0069436 A1
(43) Pub. Date: Mar. 5, 2020

(54) ARTIFICIAL INTERVERTEBRAL DISC

(71) Applicant: EXPONENTIAL MEDICAL TECHNOLOGIES (PROPRIETARY) LIMITED, Centurion (ZA)

(72) Inventors: Christopher Daniel PARKER, Gauteng (ZA); Andrew ROGINSKY, Gauteng (ZA)

(73) Assignee: Exponential Medical Technologies (Properietary) Limited, Centurion (ZA)

(21) Appl. No.: 16/348,418

(22) PCT Filed: Nov. 9, 2017
(86) PCT No.: PCT/IB2017/057011

§ 371 Date: May 8, 2019

(30) Foreign Application Priority Data
Nov. 9, 2016 (ZA) 2016/07750
Nov. 9, 2016 (ZA) 2016/07751

(51) Int. Cl. A61F 2/44

(52) U.S. Cl. CPC A61F 2/4425; A61F 2002/30253

(57) ABSTRACT

This invention concerns an artificial intervertebral disc for installation in an intervertebral space between adjacent vertebral bodies. The artificial intervertebral disc prosthesis comprises an upper plate, a lower plate and a mobile core element, which is, in use, located between the upper and lower plates. The mobile core element includes a first contoured surface that has a flexion/extension radius that is larger by a determined amount than the radius of its second contoured surface that results in an instantaneous centre of rotation below the midline of the intervertebral disc space, thereby approximating the typical instantaneous centre of rotation of a natural disc. The first contoured surface of the mobile core element has a lateral bending radius that is unequal to the value of the flexion/extension radius, thereby allowing the mobile core element to self-centre on the second surface of the upper plate when under preload. The mobile core element may further be compressible and may include a resilient element located within the mobile core element. Deformation of the resilient element may be contained to obtain an exponential increase in spring stiffness of the resilient element during compression.

### BACKGROUND TO THE INVENTION

This invention relates to an artificial intervertebral disc. In particular, but not exclusively, this invention relates to an artificial intervertebral disc comprising a compressible core.

Back pain is a common problem that affects many people at some point in their life. A common cause of back pain is degeneration of the bone surrounding the soft tissue structures of an intervertebral disc, which allow the adjacent vertebrae to move relative to one another. Another common cause of back pain is the dysfunction of an intervertebral disc, which could be as a result of wear or acute injury, for example.

Damage to an intervertebral disc often results in the destabilization of the spine, which in turn may result in alteration of a natural spacing between adjacent vertebrae. Alteration of the natural spacing between adjacent vertebrae may subject nerves that pass between these bodies to pressure.

Such pressure applied to nerves is a known cause of pain and/or nerve damage. It is accordingly important to maintain the natural spacing and mobility between adjacent vertebrae to reduce pressure applied to nerves that pass between them. In order to approximate the natural spacing and movement of a natural intervertebral disc, an artificial intervertebral disc may be implanted into a patients spine. An artificial intervertebral disc typically also limits relative motion of the adjacent vertebrae.

It is an object of this invention to alleviate at least some of the problems experienced with known artificial intervertebral discs.

It is a further object of this invention to provide an artificial intervertebral disc that will be a useful alternative to existing artificial intervertebral discs.

### SUMMARY OF THE INVENTION

In accordance with the invention there is provided an artificial intervertebral disc for installation in an intervertebral space between adjacent vertebral bodies, the disc comprising:
an upper plate having a first surface to engage with a first of the adjacent vertebral bodies and a second surface comprising a contoured, partially toroidal articulating wear surface;
a lower plate having a first surface to engage with a second of the adjacent intervertebral bodies and a second surface comprising a contoured, partially spherical articulating wear surface; and
a mobile core element which is, in use, located between the upper and lower plates such that the upper and lower plates articulate over the mobile core element;
wherein the mobile core element comprises a first contoured surface which substantially corresponds to the second surface of the upper plate and a second contoured surface which substantially corresponds to the second surface of the lower plate, wherein the first contoured surface of the mobile core element has a flexion/extension radius that is larger by a determined amount than that of the radius of the second contoured surface that results in an instantaneous centre of rotation below the midline of the intervertebral disc space, thereby approximating the typical instantaneous centre of rotation of a natural disc, and wherein the first contoured surface of the mobile core element has a lateral bending radius that is different from or unequal to the flexion/extension radius, thereby allowing the mobile core element to self-centre on the second surface of the upper plate when under preload.

In one embodiment the lateral bending radius is at least twice the value of the flexion/extension radius.

The mobile core element may comprise a first body and a second body which are moveable relative to one another between a first, uncompressed configuration and a second compressed configuration, wherein the first body and second body are arranged to restrict deformation of a resilient element located within a recess of the mobile core element as the mobile core element is compressed through movement of the first and second bodies into their compressed configuration, thereby obtaining an exponential increase in spring stiffness of the resilient element during compression.

The resilient member may be located within an enclosed volume within the mobile core element. The enclosed volume may be defined between the upper body and the lower body such that compression of the mobile core element reduces the free space within the enclosed volume, thereby containing the deformation of the resilient element.

The upper plate and mobile core element may comprise complementary shaped engagement formations which, in use, engage each other to restrict motion of the mobile core element.

The engagement formation on the upper plate may be in the form of a socket formed in the first surface of the upper plate. The socket may comprise two angled, square undercut pockets to restrict relative motion of the upper plate and core in flexion and extension. The socket may further include two peripheral lateral side walls which, in use, prevent over-translation in lateral bending

The engagement formation on the mobile core element may be in the form of a peripheral lip which is, in use, received in the socket, and in particular the undercut pockets.

The first surfaces of the upper and lower plates may each be tapered to create bevelled surfaces.

The first surfaces of the upper and lower plates carry a domed central portion.

The first surfaces of the upper and lower plates may further carry serrations. In one embodiment the serrations on the upper plate align with the serrations in the lower plate.

Each of the upper and lower plates may include a keel carrying a chamfered leading edge and a slot that extends to the base of the keel.

An artificial intervertebral disc for installation in an intervertebral space between adjacent vertebral bodies, the artificial intervertebral disc prosthesis comprising:
an upper plate having a first surface to engage with a first of the adjacent vertebral bodies and a second surface comprising a contoured, partially toroidal articulating wear surface;
a lower plate having a first surface to engage with a second of the adjacent intervertebral bodies and a second surface comprising a contoured, partially spherical articulating wear surface; and
a mobile core element which is, in use, located between the upper and lower plates such that the upper and lower plates articulate over the mobile core element;
wherein the mobile core element comprises a first contoured surface which substantially corresponds to the second surface of the upper plate and a second contoured surface which substantially corresponds to the second surface of the lower plate, wherein the first contoured surface of the mobile core element has a flexion/extension radius that is larger by a determined amount than that of the radius of the second contoured surface that results in an instantaneous centre of rotation below the midline of the intervertebral disc space, thereby approximating the typical instantaneous centre of rotation of a natural disc, and wherein the first contoured surface of the mobile core element has a lateral bending radius that is different from or unequal to the flexion/extension radius, thereby allowing the mobile core element to self-centre on the second surface of the upper plate when under preload.

In accordance with another aspect of the invention there is provided a method of approximating the typical instantaneous centre of rotation of a natural disc, the method including using an artificial intervertebral disc that has an instantaneous centre of rotation below the midline of the intervertebral disc space. The artificial intervertebral disc may be a disc in accordance with the invention.

In accordance with another aspect of the invention there is provided an intervertebral disc prosthesis comprising:
a mobile core element having upper and lower curved surfaces; and
upper and lower plates disposed about the core, each plate comprising an outer surface which engages a vertebra an inner surface shaped to slide over one of the curved surfaces of the core;
wherein the inner surface of the upper plate comprises a contoured, partially toroidal articulating wear surface;
wherein the inner surface of the lower plate comprises a contoured, partially spherical articulating wear surface;
wherein the upper and lower surfaces of the mobile core element substantially correspond to the inner surfaces of the upper and lower plates respectively;
wherein the upper surface of the mobile core element has a flexion/extension radius that is larger by a determined amount than that of the radius of its lower surface that results in an instantaneous centre of rotation below the midline of the intervertebral disc space, thereby approximating the typical instantaneous centre of rotation of a natural disc; and
wherein the upper surface of the mobile core element has a lateral bending radius that is at least twice the value of the flexion/extension radius, thereby allowing the mobile core element to self-centre on the inner surface of the upper plate when under preload.

In accordance with another aspect of the invention there is provided an artificial intervertebral disc for installation in an intervertebral space between adjacent vertebral bodies, the disc comprising:
an upper plate having a first surface to engage with a first of the adjacent vertebral bodies and a second surface comprising a contoured, partially toroidal articulating wear surface;
a lower plate having a first surface to engage with a second of the adjacent intervertebral bodies and a second surface comprising a contoured, partially spherical articulating wear surface; and
a compressible mobile core element which is, in use, located between the upper and lower plates such that the upper and lower plates articulate over the mobile core element;
wherein the mobile core element comprises a first contoured surface which substantially corresponds to the second surface of the upper plate and a second contoured surface which substantially corresponds to the second surface of the lower plate; and
wherein the compressible mobile core element comprises a first body and a second body which are moveable relative to one another between a first, uncompressed configuration and a second compressed configuration and which are arranged to restrict deformation of the resilient element as the mobile core element is compressed through movement of the first and second bodies into their compressed configuration, thereby obtaining an exponential increase in spring stiffness of the resilient element during compression.

The first body may include a recess for receiving the resilient element. The recess may be in the form of a countersunk, annular groove defining a substantially flat, inner perimetrical surface and a substantially flat, outer perimetrical surface between which the resilient element is, in use, received such that the inner perimetrical surface contacts a radially superior portion of the resilient element during compression of the mobile core element.

Alternatively, the recess may be in the form of an elliptical bore, defining a substantially flat, bottom surface and a substantially flat, perimetrical surface, in which the resilient element is, in use, received such that the perimetrical surface contacts the radial outer portion of the resilient element during compression of the mobile core element.

The second body may include a substantially annular protrusion carrying a grooved surface on which the resilient element settles, wherein the annular protrusion is received in the annular groove when the first and second bodies are moved into their compressed configuration.

Alternatively, the second body may include an elliptical protrusion carrying a matching surface on which the resilient element settles, wherein the elliptical protrusion is received in the elliptical bore when the first and second bodies are moved into their compressed configuration.

The annular groove and annular protrusion carry bearing surfaces which slide over each other as the mobile core element is compressed, thereby allowing lateral load transmission between the two bodies.

Alternatively, the elliptical bore and elliptical protrusion carry bearing surfaces which slide over each other as the mobile core element is compressed, thereby allowing lateral load transmission between the two bodies.

In one embodiment the resilient element is a ring-shaped, elastic spring component, such as an O-ring. In another embodiment the resilient element an elliptical elastic spring component, such as a silicone insert.

In one embodiment the first and second bodies may carry aligned recesses for receiving an assembly pin therein. In the preferred embodiment the pin is in the form of a polymeric, press-fit assembly pin which is locatable in the recess in the first body by press-fit.

The pin and second body may have limit stops which, in use, interacts to limit the range of axial movement of the first and second bodies relative to one another. The limit stops may be in the form of shoulders located on the pin and second body respectively.

The upper plate and mobile core element may comprise complementary shaped engagement formations which, in use, engage each other to restrict motion of the mobile core element.

The engagement formation on the upper plate may be in the form of a socket formed in the first surface of the upper plate. The socket may comprise two angled, square undercut pockets to restrict relative motion of the upper plate and core in flexion and extension. The socket may further include two peripheral lateral side walls which, in use, prevent over-translation in lateral bending

The engagement formation on the mobile core element may be in the form of a peripheral lip which is, in use, received in the socket, and in particular the undercut pockets.

The first surfaces of the upper and lower plates may each be tapered to create bevelled surfaces.

The first surfaces of the upper and lower plates carry a domed central portion.

The first surfaces of the upper and lower plates may further carry serrations. In one embodiment the serrations on the upper plate align with the serrations in the lower plate.

Each of the upper and lower plates may include a keel carrying a chamfered leading edge and a slot that extends to the base of the keel.

The resilient core element may have means for locating the resilient element in the bore of the upper body. In one embodiment the upper body and resilient element may carry complementary shaped locating formations so as to locate the resilient member with respect to the upper body. In one embodiment, the locating formations, in use, obstruct rotational movement between the resilient element and the upper body.

The upper body may carry a locating formation which protrudes into the bore and the resilient element may carry a complementary shaped recess or indentation for receiving the locating formation protruding from the upper body. The locating formation may in the form of a rectangular protrusion while the indentation may be in the form of a slot carried on an upper surface of the resilient element.

In another embodiment the resilient element may be shaped complementary to the bore such that the resilient element locates automatically within the bore when received in the bore. The resilient element is preferably dimensioned such that its outer perimetrical surface locates against the bearing surface of the upper body.

The resilient element may include an axial hole in order to allow deformation of the element during compression.

In accordance with another aspect of the invention there is provided a method of approximating the natural behaviour of an intervertebral disc in an artificial intervertebral disc by compressing a compressible mobile core of the artificial intervertebral disc while restricting the deformation of a resilient element located in the mobile core, thereby obtaining an exponential increase in spring stiffness during compression.

The method may include restricting compression of the resilient element by locating the resilient element in an enclosed space.

The method may include reducing the volume of the enclosed space during compression of the mobile core.

The method may include allowing the resilient core to expand or deform radially while compressing the resilient core axially.

The method may further include locating the resilient core inside an elliptical bore inside an upper body of the core.

In one embodiment the method includes compressing the resilient core by moving a lower body relative to the upper body.

The method may further comprise carrying the resilient element on the lower body and moving the lower body in the bore located in the upper body when compressing the resilient element.

The method may include sliding the lower body along a bearing surface of the bore inside the upper body when compressing the resilient insert.

The method may further include locating the resilient element relative to the upper body.

In one embodiment the method includes preventing rotational movement of the resilient element relative to the upper body.

The method may be carried out using the disc according to the first aspect of the invention.

In accordance with another aspect of the invention there is provided an intervertebral disc prosthesis comprising:
a mobile core element having upper and lower curved surfaces; and
upper and lower plates disposed about the core, each plate comprising an outer surface which engages a vertebra an inner surface shaped to slide over one of the curved surfaces of the core;
wherein the inner surface of the upper plate comprises a contoured, partially toroidal articulating wear surface;
wherein the inner surface of the lower plate comprises a contoured, partially spherical articulating wear surface;
wherein the upper and lower surfaces of the mobile core element substantially correspond to the inner surfaces of the upper and lower plates respectively;
wherein the upper surface of the mobile core element has a flexion/extension radius that is larger by a determined amount than that of the radius of its lower surface that results in an instantaneous centre of rotation below the midline of the intervertebral disc space, thereby approximating the typical instantaneous centre of rotation of a natural disc; and
wherein the upper surface of the mobile core element has a lateral bending radius that is different from or unequal to the flexion/extension radius, thereby allowing the mobile core element to self-centre on the inner surface of the upper plate when under preload.

### BRIEF DESCRIPTION OF THE DRAWINGS

The invention will now be described in more detail, by way of example only, with reference to the accompanying drawings in which:

FIG. 1 shows a first bottom perspective view of an artificial intervertebral disc in accordance with the invention;

FIG. 2 shows a second bottom perspective view of the artificial intervertebral disc of FIG. 1 wherein the disc is rotated through 90 degrees compared to the view of FIG. 1;

FIG. 3 shows a top perspective view of the artificial intervertebral disc of FIG. 1;

FIG. 4 shows a first cross-sectional view of the artificial intervertebral disc of FIG. 1;

FIG. 5 shows a second cross-sectional view of the artificial intervertebral disc of FIG. 1 taken perpendicularly to the cross-section of FIG. 4;

FIG. 6 shows a cross-sectional view of the artificial intervertebral disc taken along A-A as shown in FIG. 5;

FIG. 7 shows a side view of a spinal cord illustrating the instantaneous centre of rotation (ICR) of the vertebrae;

FIG. 8 shows an exploded top perspective view of a second embodiment of an artificial intervertebral disc comprising a compressible core element in accordance with the invention;

FIG. 9 shows an exploded bottom perspective view of the artificial intervertebral disc of FIG. 8 wherein the compressible core element is shown assembled;

FIG. 10 shows a front view of the compressible core element of the artificial intervertebral disc of FIG. 8;

FIG. 11 shows a first cross-sectional view of the compressible core element of the artificial intervertebral disc of FIG. 8 taken along B-B as shown in FIG. 10, wherein the core element is shown in a first, uncompressed state;

FIG. 12 shows a second cross-sectional view of the compressible core element of the artificial intervertebral disc of FIG. 8 taken along B-B as shown in FIG. 10, wherein the core element is shown in a second, compressed state;

FIG. 13 shows a load vs displacement graph indicating the exponential increase in spring stiffness obtained by compressing the compressible mobile core element from its uncompressed state to compressed state;

FIG. 14 shows an exploded top perspective view of a third embodiment of an artificial intervertebral disc comprising a compressible core element in accordance with the invention;

FIG. 15 shows an exploded bottom perspective view of the artificial intervertebral disc of FIG. 14 wherein the compressible core element is shown assembled;

FIG. 16 shows an exploded top perspective view of the compressible core element of the disc of FIG. 14;

FIG. 17 shows an exploded bottom perspective view of the compressible core element of the disc of FIG. 14;

FIG. 18 shows a front view of the compressible core element of the artificial intervertebral disc of FIG. 14;

FIG. 19 shows a first cross-sectional view of the compressible core element of the artificial intervertebral disc of FIG. 14 taken along C-C as shown in FIG. 15, wherein the core element is shown in a first, uncompressed state;

FIG. 20 shows a second cross-sectional view of the compressible core element of the artificial intervertebral disc of FIG. 14 taken along a plane perpendicular to C-C as shown in FIG. 18, wherein the core element is shown in a second, compressed state;

FIG. 21 shows a load vs displacement graph indicating the exponential increase in spring stiffness obtained by compressing the compressible mobile core element of the disc of FIG. 14 from its uncompressed state to compressed state;

FIG. 22 shows an exploded top perspective view of another embodiment of a compressible core element in accordance with the invention;

FIG. 23 shows an exploded bottom perspective view of the compressible core element of FIG. 22;

FIG. 24 shows a front view of the compressible core element of FIG. 22 wherein the core element is shown in a first, uncompressed state;

FIG. 25 shows a cross-sectional view of the compressible core element of FIG. 22 taken along A-A as shown in FIG. 22, wherein the core element is shown in a first, uncompressed state;

FIG. 26 shows a front view of the compressible core element of FIG. 22 wherein the core element is shown in a second, compressed state; and

FIG. 27 shows a cross-sectional view of the compressible core element of FIG. 22 taken along D-D as shown in FIG. 26, wherein the core element is shown in a second, compressed state.

### DESCRIPTION OF THE ILLUSTRATED EMBODIMENT

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings and are thus intended to include direct connections between two members without any other members interposed therebetween and indirect connections between members in which one or more other members are interposed therebetween. Further, “connected” and “coupled” are not restricted to physical or mechanical connections or couplings. Additionally, the words “lower”, “upper”, “upward”, “down” and “downward” designate directions in the drawings to which reference is made. The terminology includes the words specifically mentioned above, derivatives thereof, and words or similar import. It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Referring to the drawings, in which like numerals indicate like features, a non-limiting example of a first embodiment of an artificial intervertebral disc in accordance with the invention is generally indicated by reference numeral 10. The artificial intervertebral disc 10 could also be referred to as an intervertebral disc prosthesis.

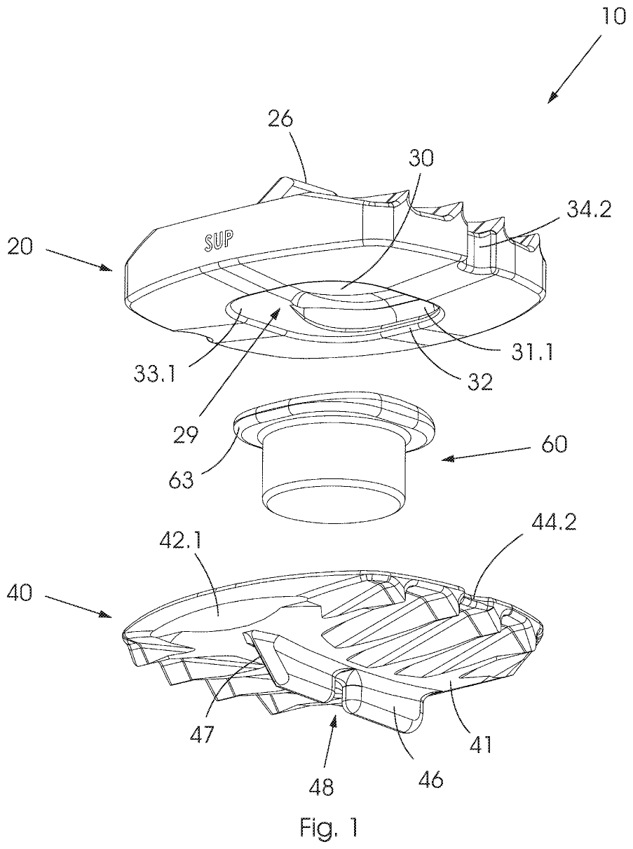

FIGS. 1 to 3 show exploded perspective views of the artificial intervertebral disc 10 in accordance with the invention. The intervertebral disc 10 includes a first plate 20, a second plate 40 and a mobile core element 60 in use located between the first and second plates. The first plate 20 and second plate 40 are also referred to as upper and lower plates due to their relative position to each other when a patient who has received the artificial intervertebral disc 10 is in up upright position. In the accompanying drawings the intervertebral disc 10 is shown in exploded view wherein the upper plate 20, lower plate 40 and mobile core element 60 are disengaged from one another so that the features of these components are clearly visible.

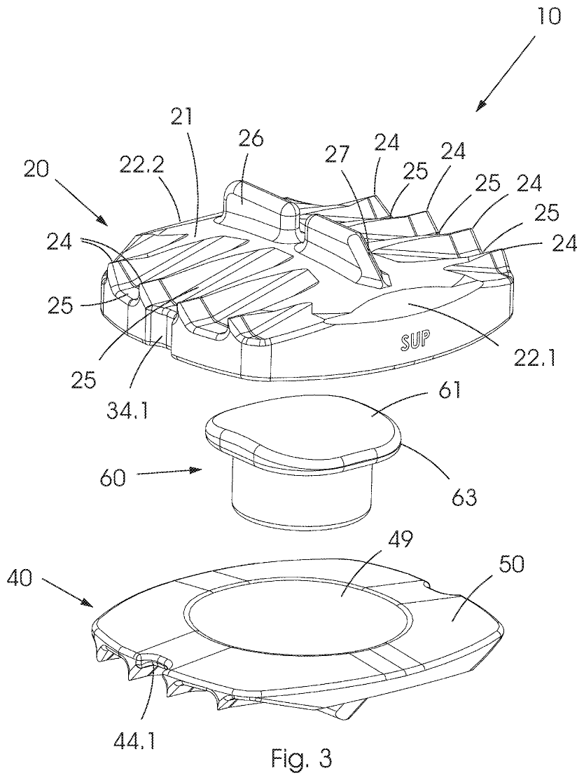

The upper plate 20 of the intervertebral disc 10 has a first surface 21 for engagement with a first adjacent vertebral body of a patient's spine. In the illustrated embodiment the upper plate 20 has a substantially domed, centrally raised first surface 21. The first surface 21 of the upper plate is tapered to create two bevelled surfaces 22.1 and 22.2 on opposed sides of the upper plate 20. The upper plate 20 further carries a number of angle serrations 23 defining ridges 24 and grooves 25, located between the ridges. Best seen in FIG. 3, the upper plate 20 carries two sets of serrations 23, which are located on opposite sides of the upper plate. The serrations 23 are configured such that the two sets of serrations on the opposed sides of the upper plate are aligned. In other words, the ridges 24 of the first set of ridges on the one side of the upper plate align with the ridges of the second set located on the opposite side of the upper plate. The grooves 25 are, accordingly, similarly aligned.

Still referring to FIG. 3, it can be seen that the upper plate 20 further has a locating formation in the form of a keel 26. In the illustrated embodiment the keel 26 has a chamfered, leading edge 27, which is also referred to as the leading edge. A slot 28 is formed in the keel 26 and extends substantially to the base of the keel, i.e. to the first surface 21.

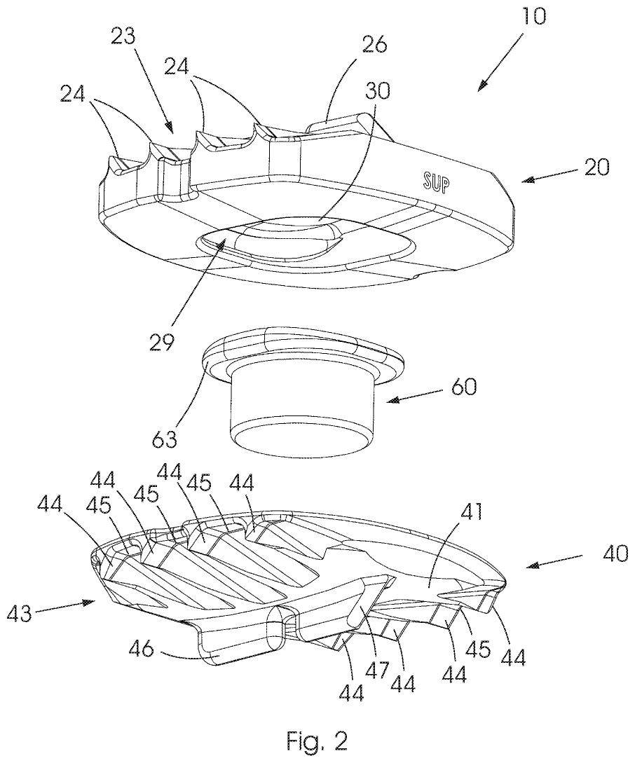

Referring now in particular to FIGS. 1 and 2, the upper plate 20 of the intervertebral disc 10 has a receiving formation for receiving the mobile core element 60, in use. In the illustrated embodiment the receiving formation is in the form of a socket 29 carried in the bottom surface or underside of the upper plate 20. The socket 29 defines a second surface 30 in the form of a contoured, partially toroidal, articulating wear surface. In view of the configuration of the intervertebral disc 10 the first and second surfaces 21, 30 can also be referred to as exterior and interior surfaces. Alternatively, the first and second surfaces 21, 30 may also be referred to as upper and lower surfaces respectively due to their operative relative positions.

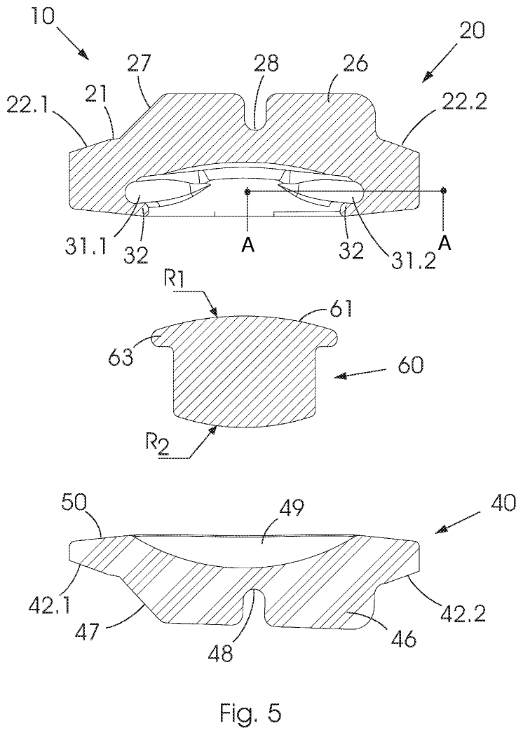

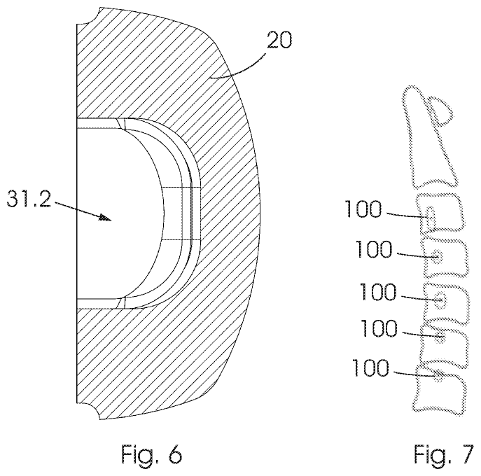

The upper plate 20 further has two retaining formations in the form of internal pockets 31.1 and 31.2. The pockets 31.1 and 31.2 are defined by the socket 29. Probably best seen in FIGS. 5 and 6, the pockets 31.1 and 31.2 are angled undercuts that restrict motion of the mobile core element 60 when received in the socket 29. The undercuts 31.1 and 31.2 create retaining formations or lips 32 for retaining complementary shaped portions of the mobile core therein, thereby restricting movement of the mobile core when located in the socket 29. In the detail cross-sectional view of FIG. 6 it can be seen that the undercuts 31.1 and 31.2 are substantially square with rounded corners. The illustrated embodiment of the upper plate 20 has two undercuts 31.1 and 31.2 which are again located substantially opposite each other. The undercuts 31.1 and 31.2 are substantially aligned with the bevelled or chamfered edges 22.1 and 22.2. Running between the undercuts 31.1 and 31.2 are two peripheral lateral side walls 33.1 and 33.2.

The undercuts are shaped complementary to the mobile core element 60. More about this is said below.

The second or lower plate 40 of the intervertebral disc 10 also has a first surface 41 for engagement with a second adjacent vertebral body of a patient's spine. In the illustrated embodiment the lower plate 40 has a substantially domed, centrally raised first surface 41. The first surface 41 of the lower plate is again tapered to create two bevelled surfaces 42.1 and 42.2 on opposed sides of the lower plate 40. Similarly to the upper plate 20, the lower plate carries a number of angle serrations 43 defining ridges 44 and grooves 45, located between the ridges. The serrations 43 are arranged similarly to those of the upper plate 20 and therefore will not be described in detail again.

Best seen in FIGS. 1 and 2, the lower plate 40 also has a locating formation in the form of a keel 46, which is substantially similar to the keel 26 of the upper plate 20. The keel 46 again has a chamfered edge 47, which is again referred to as the leading edge. A slot 48 is formed in the keel 46 and extends substantially to the base of the keel, i.e. to the first surface 41.

In use, the upper and lower plates 20, 40 are oriented so that their serrations 23, 43 and keels 26, 46 are aligned thereby to facilitate engagement with the first and second adjacent vertebras respectively. The keels 26 and 46 extend or face the same direction. In use, the leading edges of the keels 27, 47 cut into the vertebral bodies. The serrations 23 and 43 on the other hand face substantially opposite directions. The upper 20 and lower 40 plates each carries locating formations which are again aligned with one another, in use. The locating formations in the upper end plate 20 are indicated by the reference signs 34.1 and 34.2, while the locating formations in the lower end plate 40 are indicated by the reference signs 44.1 and 44.2.

Referring now in particular to FIGS. 3 to 5, the lower plate 40 of the intervertebral disc 10 again has a receiving formation for receiving a portion of the mobile core element 60, in use. In the illustrated embodiment the receiving formation is in the form of a recess 49 carried on a second surface 50, which is, in use, at a top or upper surface of the lower plate 40. The second surface 50, and in particular the recess 49, defines a contoured partially spherical articulating wear surface which, in use, engages a complementary shaped wear surface of the mobile core element 60.

As shown in the accompanying drawings, the mobile core element 60 is, in use, located between the upper plate 20 and the lower plate 40, such that the upper and lower plates articulate over the mobile core when implanted in the patient's intervertebral disc space.

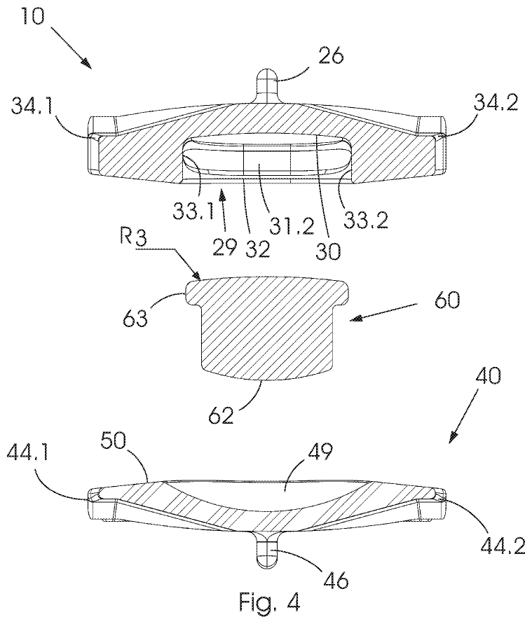

Referring in particular to FIGS. 4 and 5, the mobile core element 60 has a first contoured surface 61 which is complementary shaped to the internal surface 30 of the socket 29 of the upper plate 20. Similarly, the mobile core element 60 has a second contoured surface 62 which is complementary shaped to the second surface 50, and in particular the recess 49 defined by the surface 50, of the lower plate 20.

The first contoured surface 61 of the mobile core element 60 comprises a flexion/extension radius R₁ (FIG. 5) that is larger by a determined amount than that of the inferior articulating spherical radius R₂ of the second contoured surface 62, wherein the predetermined amount is such that its instantaneous centre of rotation (ICR) is lowered to below the midline of the intervertebral disc space, thereby mimicking or approximating the typical ICR of a natural disc as determined by Penning and Wilmink (1987) and illustrated in FIG. 7. The typical ICR of a natural disc is indicated by the numeral 100 in FIG. 7.

Referring now to FIG. 4, the first surface 61 of the mobile core further has a lateral bending radius R₃ that is different from or unequal to the first surface's flexion/extension radius R₁. This configuration allows the mobile core element 60 to self-centre on the second, inner surface 30 of the upper plate 20 when under preload. It is envisaged that the lateral bending radius R₃ could be at least twice the value of the first surface's flexion/extension radius R₁.

This difference in the flexion/extension and lateral bending radii R₁, R₃ allows for customisation of the rate of translation in either of those motions, i.e. flexion/extension and lateral bending. It has further been found that the combination of the lateral side walls 33.1, 33.2 and a larger flexion/extension radius R₃ allows for a more natural combination of translations, i.e. less translation in lateral bending than in flexion/extension.

The mobile core element 60 further has a retaining or engaging formation in the form of a continuous peripheral lip 63 carried at an end which is, in use, its upper end. The lip 63 is shaped to engage the complementary shaped engagement formations in the form of the undercut pockets 31.1, 31.2 of the upper plate 20 in order to restrict relative motion between the upper plate 20 and the mobile core element 60 in flexion and extension. In use, the lip 63 further engages the side walls 33.1 and 33.2 of the socket 29 of the upper plate 20 in order to prevent over-translation in lateral bending.

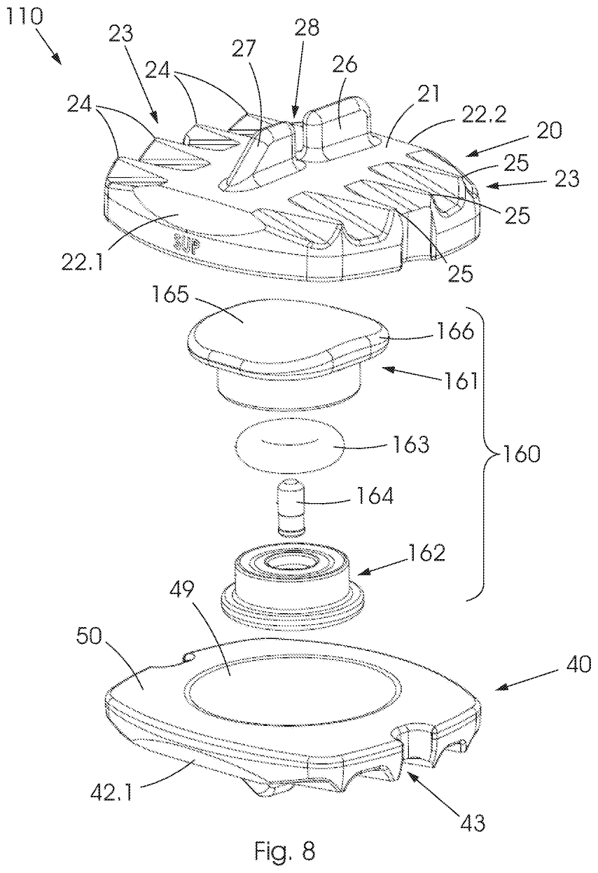

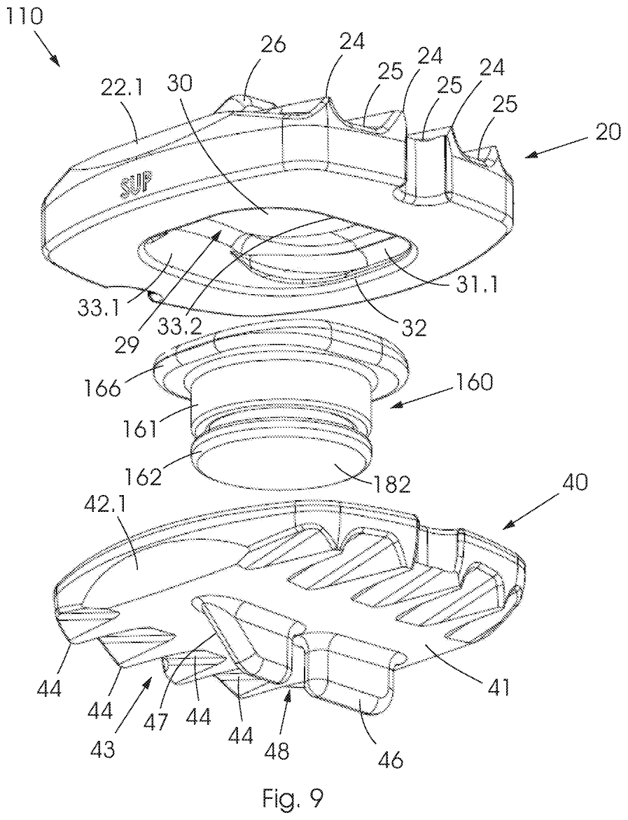

Turning now to FIGS. 8 to 13, in which like numerals again indicate like features, a non-limiting example of a second embodiment of an artificial intervertebral disc in accordance with the invention is generally indicated by reference numeral 110. The second embodiment of the invention is substantially similar to the first embodiment apart from that the second embodiment includes a compressible mobile core.

FIG. 8 shows an exploded perspective views of the artificial intervertebral disc 110 in accordance with the invention. The first and second plates of the second disc 110 are substantially identical to the first 20 and second 40 plates of the disc 10 and will therefore not be described again in any detail. However, in this second embodiment the mobile core is in the form of a mobile, compressible core element 160 which is, in use, located between the first 20 and second 40 plates. Similarly to the first embodiment of the invention, the upper 20 and lower 40 plates articulate over the mobile core 160 when implanted in the patient's spine.

Referring still to FIG. 8, the mobile compressible core element 160 comprises a first or upper body 161, a second or lower body 162, a resilient, compressible element 163 and a locating pin 164. The upper body 161 of the mobile core element 160 carrying a first contoured surface 165 in the form of a superior facing, toroidal, low-friction wear surface which is complementary shaped to the internal surface 30 of the socket 29 of the upper plate 20. In use, the wear surface 165 engages the second surface of the upper plate 20.

The upper body 161 of the mobile core element 160 further has a retaining or engaging formation in the form of a continuous peripheral lip 166 carried at an end which is, in use, its upper end. The lip 166 is shaped to engage the complementary shaped engagement formations in the form of the undercut pockets 31.1, 31.2 of the upper plate 20 in order to restrict relative motion between the upper plate 20 and the mobile core element 160 in flexion and extension. In use, the lip 166 further engages the side walls 33.1 and 33.2 of the socket 29 of the upper plate 20 in order to prevent over-translation in lateral bending.

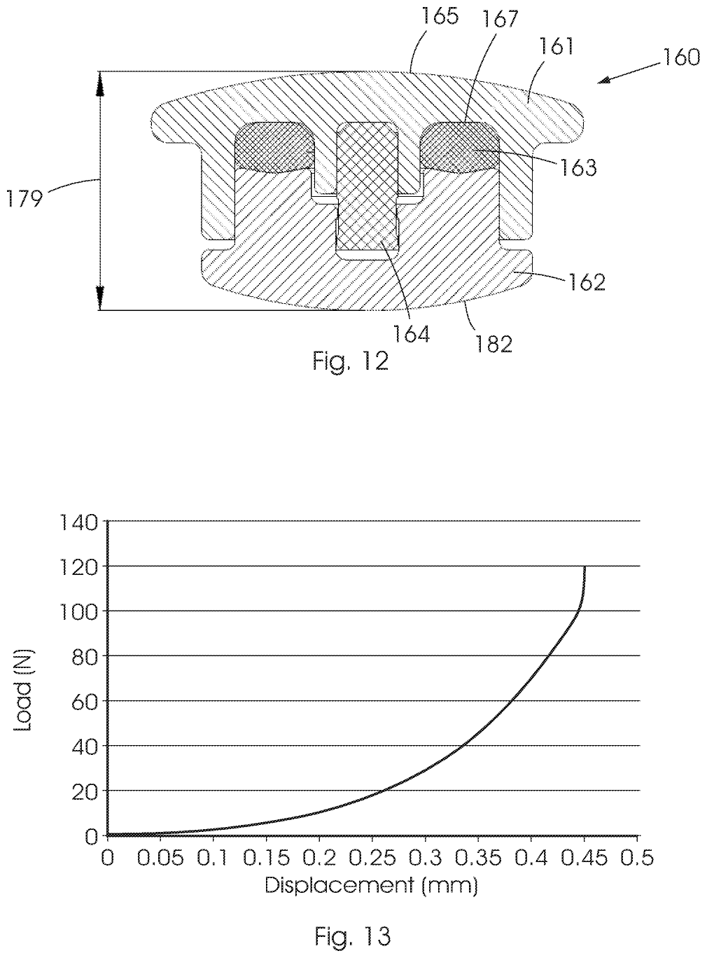

Turning now to FIG. 11, the upper body 161 of the mobile core element 160 has recess for receiving the resilient element 163. In the illustrated embodiment the recess, for receiving the resilient element, is in the form of a countersunk groove 167. The resilient element is preferably a ring-shaped, elastic spring component, such as an O-ring, for example. It should be understood that the word spring does not describe any particular shape of elastic or resilient component. Instead, the word spring is used to describe an elastic device that can be deformed and returns to its former shape when released. Accordingly, in order to receive the resilient element the countersunk groove is annular in shape and has a substantially flat, inner perimetrical surface 168 and a substantially flat, outer perimetrical surface 169. The inner surface 168 contacts a radially superior portion of the resilient element 163 when the mobile core element 160 is in its compressed configuration as shown in FIG. 12. The outer perimetrical surface 169, in turn, acts as a circumferential sliding surface when compressing the mobile core element by moving the upper and lower bodies 161, 162 towards one another. As shown in FIGS. 11 and 12, the lower body 162 of the mobile core element 160 carries an outer perimetrical sliding surface 170 which is complementary shaped to the sliding surface 169 carried by the upper body 161. The outer sliding surface 170 of the lower body 162 is defined by a substantially annular protrusion 171, which is received in the countersunk groove 167 when the mobile core element 160 is compressed. As shown in FIG. 12 in particular, the resilient element is compressed within the volume created by the countersunk groove 167 and the annular protrusion 171 when the mobile core element 160 is compressed. The annular protrusion 171 carries a tapered or grooved seating surface 172 so as to self-centre the resilient element 163 on the protrusion.

The upper and lower bodies 161 and 162 carry aligned recesses 173 and 174 respectively for receiving the pin 164 therein. In the preferred embodiment the pin 164 is in the form of a polymeric, press-fit assembly pin which is locatable in the recess 175 by press-fit. As shown in FIG. 11, the recess 174 in the lower body 162 has an undercut 175 that creates a locating formation 176 in the form of an annular shoulder. A portion 177 of the pin 164 is cut away so as to create a corresponding locating formation 178 carried by the pin. The locating formation 178 is again in the form of a shoulder and interacts with the shoulder 176 in the recess 174 to act as a limit stop. In other words, the shoulders 176 and 178 define a predetermined range of allowable movement between the upper and lower bodies 161 and 162.

In the uncompressed configuration of the mobile core element 160 of FIG. 11, the shoulders 176 and 178 abut each other to prevent any further axial movement of the upper and lower bodies 161 and 162 away from each other. In this configuration of the mobile core element 160 the only allowable axial movement between the upper and lower bodies 161, 162 is towards each other, i.e. in a direction to compress the resilient element 163. When the mobile core element 160 is compressed by compressing the O-ring 163, the pin 164 moves along and deeper into the recess 174 of the lower body 162 as shown in the compressed configuration of FIG. 12.

In this embodiment of the invention, the resilient element acts as a limit stop when compressing the mobile core element 160. As can be seen in FIG. 12, during compression the resilient element is deformed in a radial or lateral direction until the volume between the sidewalls of the recess 167 in the upper body 161 and the annular protrusion 171 is substantially fully occupied by the resilient element. In other words, during compression the resilient element is deformed until the bottom of the recess 167, the inner and outer perimetrical surfaces 168, 169 and the grooved surface 172 restrict any further deformation, thereby making the resilient element substantially incompressible. The limit stops described above act as primary compressive displacement limits to restrict the axial displacement allowed between the upper and lower bodies 161, 162.

Apart from its function described above, the elastic resilient element 163 is also used to determine the mid-operating range height 179 (FIG. 12) after implantation of the disc 110 between adjacent vertebrae of the patient. After implantation the resilient element 163 is compressed within the groove 167 under a preload, resulting in the mobile core's mid-operating range height 179.

The elastic resilient element 163 is further used to approximate the non-linear stiffness profile of a natural intervertebral disc. As mentioned above, under compression the elastic resilient element 163 expands laterally, thereby increasingly taking up the volume of the groove 167. As a result of the lateral expansion being restrained by the inner and outer perimetrical surfaces 168 and 169 there is a resulting exponential increase in the stiffness of the elastic resilient element 163. The exponential increase in spring stiffness of the resilient element is illustrated in FIG. 13.

The mobile core element 160 further comprises a secondary compressive displacement limit defined by a surface 180 on the upper body 161 and a shoulder 181 carried on the lower body 162.

Similarly to the upper body 161, the lower body 162 carries a second contoured surface 182 of the mobile core element 160 which is complementary shaped to the second surface 50, and in particular the recess 49 defined by the surface 50, of the lower plate 20. In the illustrated embodiment the second surface 182 is an inferior facing spherical low-friction surface.

It should be understood that the configuration of the upper and lower bodies 161 and 162 of the mobile core element 160, and in particular the tight fit created by the inner and outer bearing surfaces 169 and 170, allows for lateral load transmission between the two bodies 161, 162.

The first contoured surface 165 of the mobile core element 160 comprises a flexion/extension radius R₁ (FIG. 11) that is larger by a determined amount than that of the inferior articulating spherical radius R₂ of the second contoured surface 82, wherein the predetermined amount is such that its instantaneous centre of rotation (ICR) is lowered to below the midline of the intervertebral disc space, thereby approximating the typical ICR of a natural disc as determined by Penning and Wilmink (1987).

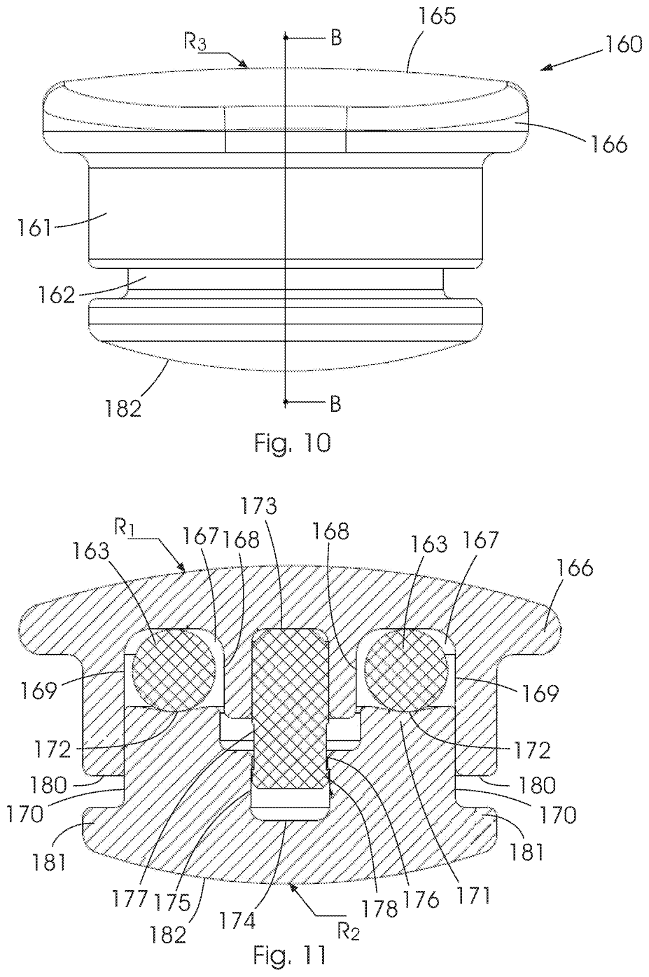

The first contoured surface 165 (FIG. 10) further has a lateral bending radius R₃ that is different from or unequal to the first surface's flexion/extension radius R₁. This configuration allows the mobile core element 160 to self-centre on the second, inner surface 30 of the upper plate 20 when under preload. It is again envisaged that the lateral bending radius R₃ could be at least twice the value of the first surface's flexion/extension radius R₁.

This difference in the flexion/extension and lateral bending radii R₁, R₃ allows for customisation of the rate of translation in either of those motions, i.e. flexion/extension and lateral bending. It has further been found that the combination of the lateral side walls 33.1, 33.2 and a larger flexion/extension radius R₃ allows for a more natural combination of translations, i.e. less translation in lateral bending than in flexion/extension.

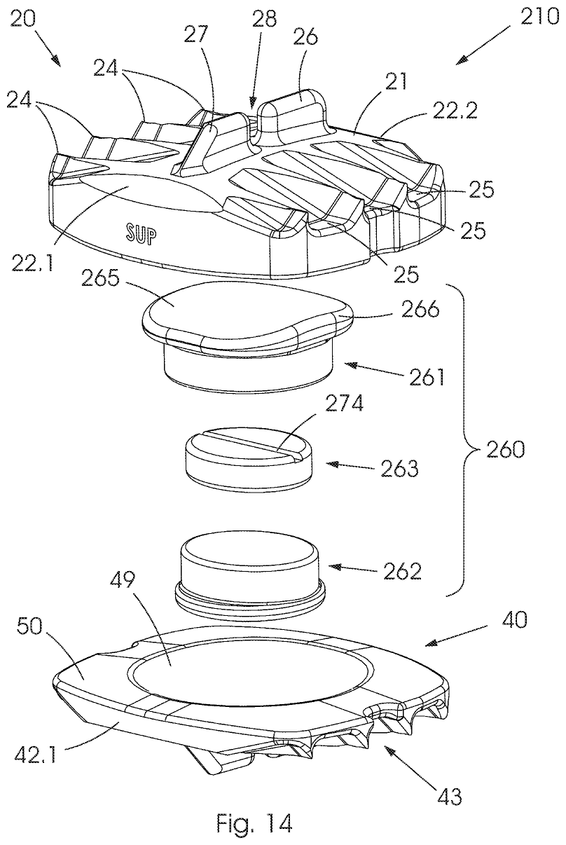

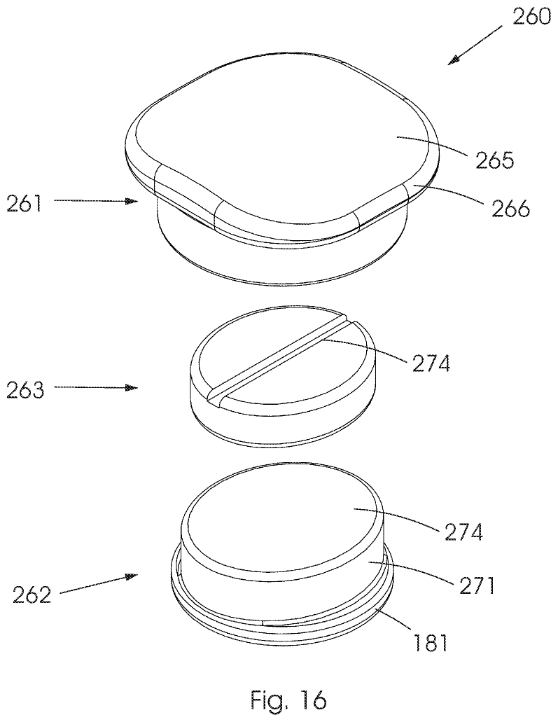

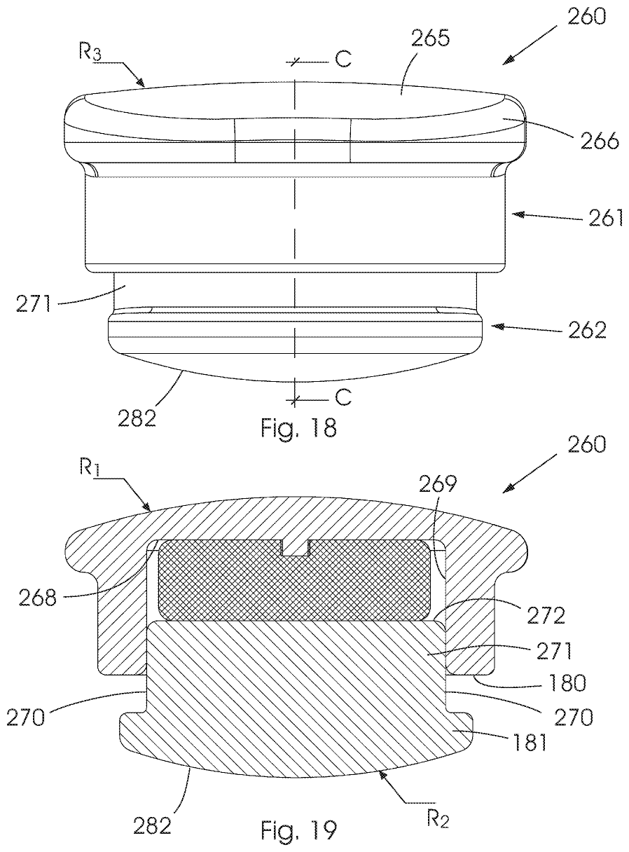

Referring now to FIGS. 14 to 21 of the drawings, a non-limiting example of a third embodiment of an artificial intervertebral disc in accordance with the invention is generally indicated by reference numeral 210. Again, like numerals indicate like features. The third embodiment of the invention is substantially similar to the second embodiment apart from the structure of the compressible mobile core, and in particular the resilient element and internal design of the mobile core interacting with the resilient element. Again the upper and lower plates of this third embodiment are substantially identical to the upper 20 and lower 40 plates of the first embodiment of the disc 10. Accordingly, only the compressible mobile core of this third embodiment will be described in detail. In FIGS. 14 to 21 the mobile compressible core is indicated by the numeral 260.

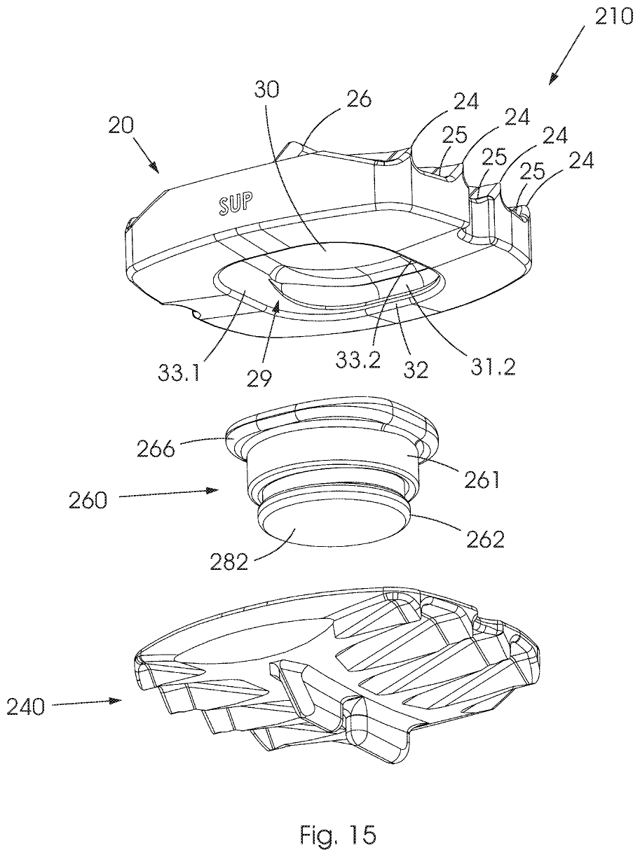

In FIGS. 14 and 15 it can be seen that the mobile compressible core element 260 comprises a first or upper body 261, a second or lower body 262 and a resilient, compressible element 263. Again, the upper body 261 of the mobile core element 260 carries a first contoured surface 265 in the form of a superior facing, toroidal, low-friction wear surface which is complementary shaped to the internal surface 30 of the socket 29 of the upper plate 20. In use, the wear surface 265 engages the second surface of the upper plate 20.

Similarly to the second embodiment of the disc 110, the upper body 261 of the mobile core element 260 has a retaining or engaging formation in the form of a continuous peripheral lip 266 carried at an end which is, in use, its upper end. The lip 266 is shaped to engage the complementary shaped engagement formations in the form of the undercut pockets 31.2, 31.2 of the upper plate 20 in order to restrict relative motion between the upper plate 20 and the mobile core element 260 in flexion and extension. In use, the lip 266 further engages the side walls 33.1 and 33.2 of the socket 29 of the upper plate 20 in order to prevent over-translation in lateral bending.

Referring to FIG. 15, in this second illustrated embodiment of the disc 210 the resilient element 263 is an elliptical, elastic spring component. It should be understood that the word spring does not describe any particular shape of elastic or resilient component. Instead, the word spring is used to describe an elastic device that can be deformed and returns to its former shape when released. In this embodiment the resilient element 263 is substantially in the form of a short, elliptical solid cylinder. Although not limited to any specific material, it is envisaged that the resilient element 263 could be made from silicone.

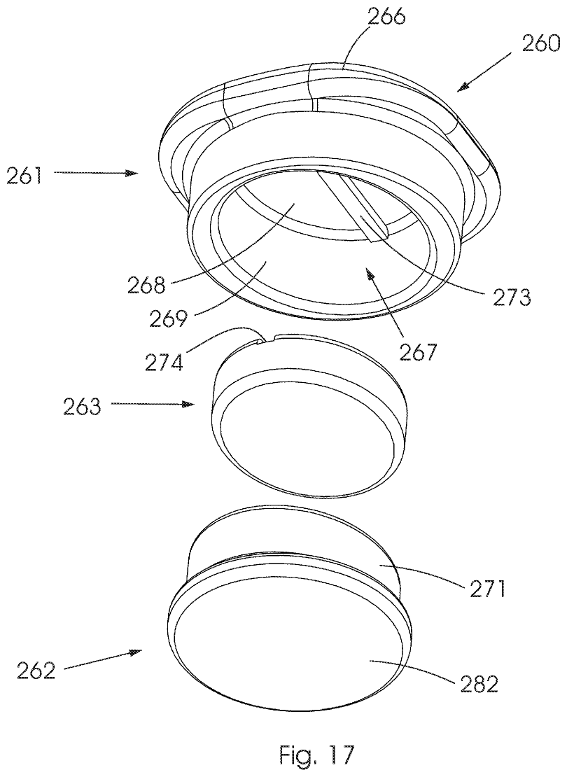

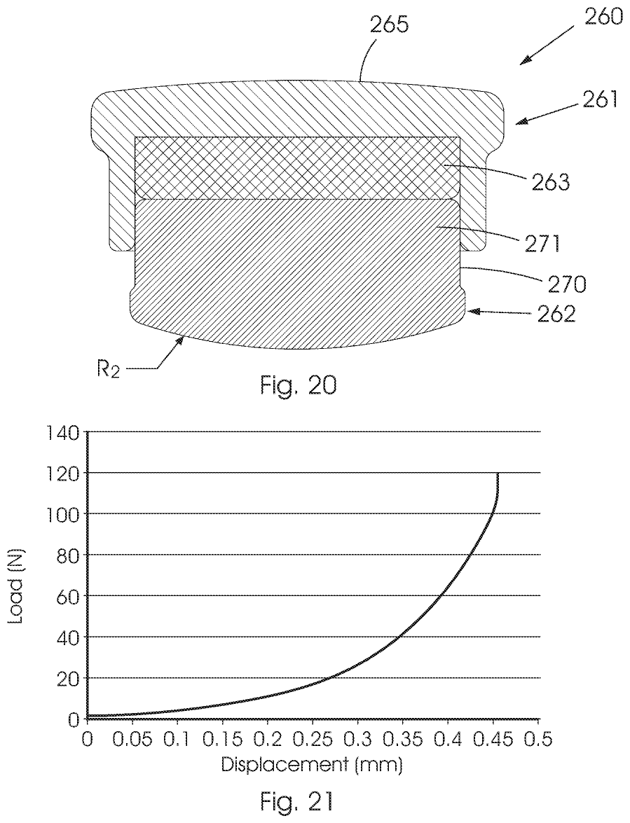

The upper body 261 of the mobile core element 260 is shaped so as to receive the resilient element at least partially. Probably best seen in FIGS. 17, 19 and 20, the upper body 261 has a recess in the shape of an elliptical bore 267 in which the resilient element 263 is received when the core 260 is assembled. The bore 267 has a substantially flat end or bottom surface 268 and a substantially flat perimetrical surface 269. The bottom surface 268 contacts the radial outer portion of the resilient element 163 when the mobile core element 260 is in its compressed configuration as shown in FIG. 20. The perimetrical surface 269, in turn, acts as a circumferential sliding surface when compressing the mobile core element by moving the upper and lower bodies 261, 262 towards one another. As shown in FIGS. 19 and 20, the lower body 262 of the mobile core element 260 carries an outer perimetrical sliding surface 270 which is complementary shaped to the sliding surface 269 carried by the upper body 261. The outer sliding surface 270 of the lower body 262 is defined by a substantially elliptical protrusion 271, which is received in the elliptical bore 167 when the mobile core element 260 is compressed. As shown in FIG. 20 in particular, the resilient element is compressed within the volume created by the elliptical bore 267 and the elliptical protrusion 271 when the mobile core element 260 is compressed. It should be understood that the resilient element is carried on an upper surface 272 of the elliptical protrusion 271.

The resilient core element 260 further has means for locating the resilient element 263 in the bore 267 of the upper body 261. The upper body 261 and resilient element 263 carry complementary shaped locating formations for locating the resilient element 263 inside the bore 267. In this second illustrated embodiment the upper body 261 carries a locating formation 273, which protrudes into the bore 267, and the resilient element 263 carries a complementary shaped recess or indentation 274 for receiving the locating formation 273. As shown in FIGS. 17 and 19, the locating formation 273 is in the form of a rectangular protrusion while the indentation 274 is in the form of a slot carried on an upper surface of the resilient element. It should be understood that the locating formations 273, 274 are not limited to any particular arrangement or shape, and could take various shapes, for example. The locating formations serve to locate the resilient member with respect to the upper body.

Similarly to the second embodiment of the disc 110, the resilient element 263 acts as a limit stop when compressing the mobile core element 260. As can be seen in FIG. 20, during compression the resilient element 263 is deformed in a radial or lateral direction until the volume between the sidewalls of the recess 267 in the upper body 261 and the elliptical protrusion 271 is substantially fully occupied by the resilient element. In other words, during compression the resilient element 263 is deformed until the bottom of the recess 267, the perimetrical surface 269 and the upper surface 272 of the protrusion 271 restrict any further deformation, thereby making the resilient element substantially incompressible. The limit stop described above act as a primary compressive displacement limit to restrict the axial displacement allowed between the upper and lower bodies 261, 262.

Similarly to mobile core 60 of the second embodiment of the disc 110, the mobile core element 160 also comprises a compressive displacement limit defined by a surface 180 on the upper body 161 and a shoulder 181 carried on the lower body 162. The displacement limit, and in particular the surface 180 and shoulder 181 act as a limit stop, preferably a secondary limit stop.

Similarly to the upper body 261, the lower body 262 carries a second contoured surface 282 of the mobile core element 260 which is complementary shaped to the second surface 50, and in particular the recess 49 defined by the surface 50, of the lower plate 20. The second surface 282 is an inferior facing spherical low-friction surface. This applies to all of the illustrated embodiments such that the second surface 62, 182 of the first and second embodiments is also an inferior facing spherical low-friction surface.

As mentioned above with reference to the second embodiment of the disc 110, the configuration of the upper and lower bodies 261 and 262 of the mobile core element 260, and in particular the tight fit created by the inner and outer bearing surfaces 269 and 270, allows for lateral load transmission between the two bodies 261, 262.

The configuration of the flexion/extension radius R₁ (FIG. 12), the articulating spherical radius R₂ and the lateral bending radius R₃ is identical to that of the mobile core 160 of the second embodiment of the disc 110. Accordingly, it will not be described again.

The resilient element 263 is again used to approximate the non-linear stiffness profile of a natural intervertebral disc. As mentioned above, under compression the resilient element 263 expands laterally, thereby increasingly taking up the volume of the bore 267. As a result of the lateral expansion being restrained by the perimetrical surface 269 there is a resulting exponential increase in the stiffness of the resilient element 263. The exponential increase in spring stiffness of the resilient element 263 is illustrated in FIG. 21.

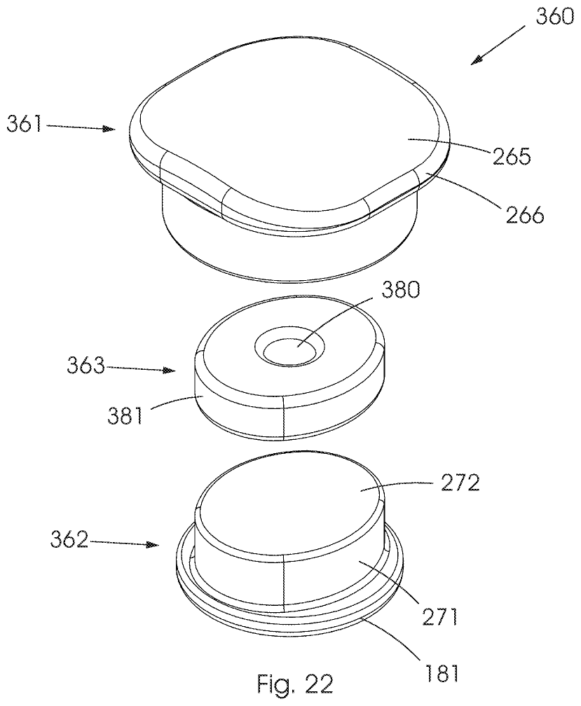

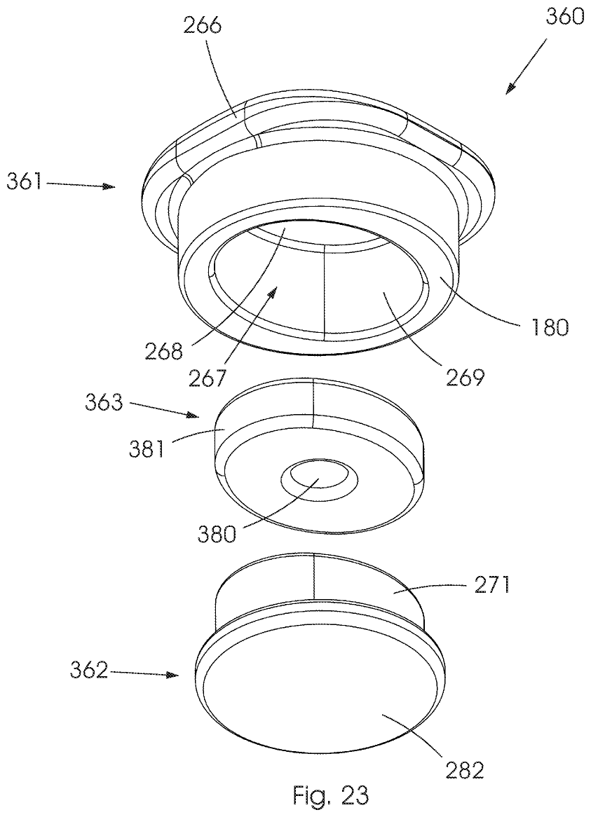

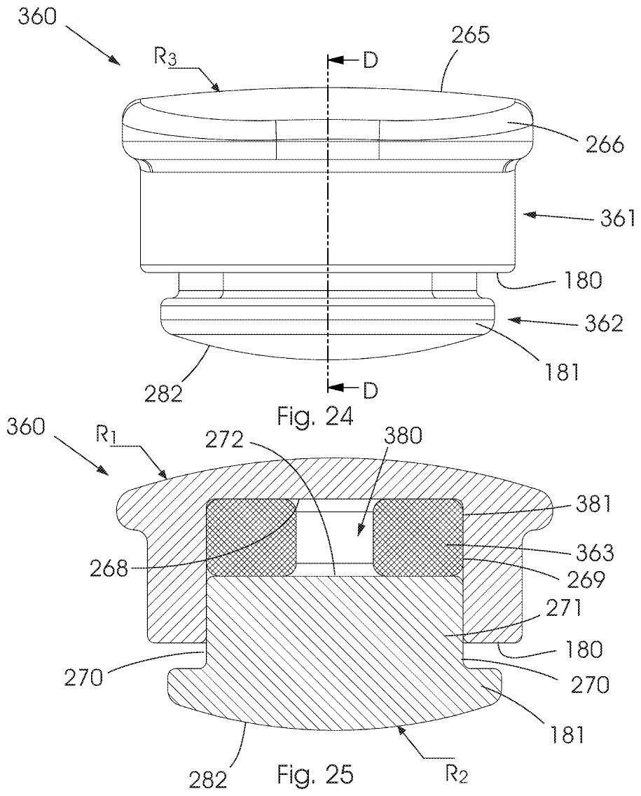

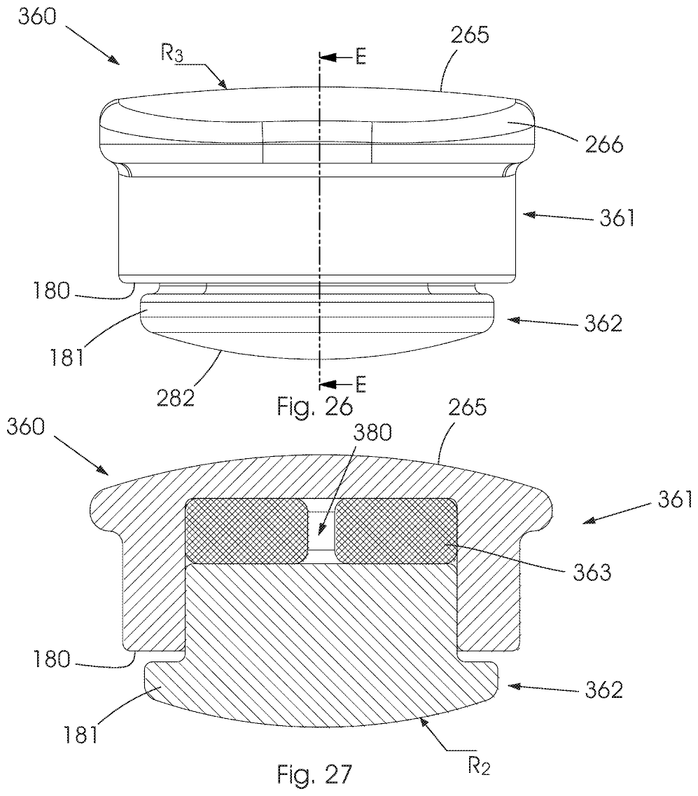

An alternative embodiment of the compressible mobile core for use in the discs 110 and 210 is shown in FIGS. 22 to 27. In these figures the mobile core is indicated by the numeral 360. The mobile core 360 again includes an upper body 361, a lower body 362 and a resilient element 361. The mobile core 360 is substantially similar to core 260 apart from the design of the resilient element. Instead of carrying locating formations the resilient element 363 locates using the surface 269 of the upper body 361. The resilient element 363 is shaped complementary to the shape of the bore 267. The resilient element is further dimensioned such that its outer perimetrical surface 381 locates against the bearing surface 269.

In the core 360 the resilient element 363 carries an axial hole 380 in order to allow deformation of the element during compression. In this particular embodiment the axial hole 380 is cylindrical in shape. It should however be clear that the invention is not limited to this particular shape and could take a variety of different shapes. As shown in FIG. 27, the resilient element 363 deforms radially inward under compression. In order words, the resilient element deforms into the free space within enclosed volume created by the axial hole 380.

The advantage of this design of the resilient element 363 is that it locates automatically within the bore 267 when received in the bore.

Similarly to the resilient elements 163 and 263, the element 363 is again used to approximate the non-linear stiffness profile of a natural intervertebral disc. As mentioned above, under compression the resilient element 363 expands laterally, thereby increasingly taking up the volume of the bore 267. As a result of the lateral expansion being restrained by the perimetrical surface 269 there is a resulting exponential increase in the stiffness of the resilient element 363. An exponential increase in spring stiffness of the resilient element 363 is again similar to that illustrated in FIG. 21.

The use of the discs 110, 210 in accordance with the invention therefore provides a method of containing the expansion of the resilient element 163, 263, 363 during compression. By locating the resilient element in an enclosed volume the expansion of the resilient element is limited to the free, unoccupied space in the enclosed volume. It should further be understood that the free space within the enclosed volume is reduced as the core 160, 260, 360 is compressed. By reducing the free space through compression of the core 160, 260, 360 a limit is reached where the resilient element 163, 263, 363 occupies the enclosed volume fully, thereby preventing further compression of the core 160, 260, 360. The expansion of the resilient element 163, 263, 363 is therefore contained by controlling the available free space.

The elastic resilient element 163, 263, 363 is also used to determine the mid-operating range height 179 (FIG. 12) after implantation of the disc 110, 210 between adjacent vertebrae of the patient. After implantation the resilient element 163, 263, 3633 is compressed under a preload, resulting in the mobile core's mid-operating range height 179.

Another advantage of the enclosed volume in which the resilient element 163, 263, 363 is located is that debris is contained within this volume. As a result of the enclosed volume the core 160, 260, 360, debris is trapped so as to prevent it from migrating out of the disc 110, 210 and into the patient's body. It should be understood that the bearing surfaces 169, 170; 269, 270 create a sealing arrangement or seal so as to seal off the enclosed volume.

It will be appreciated that the above description only provides some embodiments of the invention and that there may be many variations without departing from the spirit and/or the scope of the invention. It is easily understood from the present application that the particular features of the present invention, as generally described and illustrated in the figures, can be arranged and designed according to a wide variety of different configurations. In this way, the description of the present invention and the related figures are not provided to limit the scope of the invention but simply represent selected embodiments.

The skilled person will understand that the technical characteristics of a given embodiment can in fact be combined with characteristics of another embodiment, unless otherwise expressed or it is evident that these characteristics are incompatible. Also, the technical characteristics described in a given embodiment can be isolated from the other characteristics of this embodiment unless otherwise expressed.

1. An artificial intervertebral disc for installation in an intervertebral space between adjacent vertebral bodies, the artificial intervertebral disc prosthesis comprising:
an upper plate having a first surface to engage with a first of the adjacent vertebral bodies and a second surface comprising a contoured, partially toroidal articulating wear surface;
a lower plate having a first surface to engage with a second of the adjacent intervertebral bodies and a second surface comprising a contoured, partially spherical articulating wear surface; and
a mobile core element which is, in use, located between the upper and lower plates such that the upper and lower plates articulate over the mobile core element;
wherein the mobile core element comprises a first contoured surface which substantially corresponds to the second surface of the upper plate and a second contoured surface which substantially corresponds to the second surface of the lower plate, wherein the first contoured surface of the mobile core element has a flexion/extension radius that is larger by a determined amount than that of the radius of the second contoured surface that results in an instantaneous centre of rotation below the midline of intervertebral disc space, thereby approximating the typical instantaneous centre of rotation of a natural disc, and wherein the first contoured surface of the mobile core element has a lateral bending radius that is unequal to the value of the flexion/extension radius, thereby allowing the mobile core element to self-centre on the second surface of the upper plate when under preload; and
wherein the mobile core element comprises a first body and a second body which are moveable relative to one another between a first, uncompressed configuration and a second compressed configuration, and wherein the first body and second body are arranged to restrict deformation of a resilient element located within the mobile core element as the mobile core element is compressed through movement of the first and second bodies into their compressed configuration, thereby obtaining an exponential increase in spring stiffness of the resilient element during compression.

2. (canceled)

3. An artificial intervertebral disc according to claim 1, wherein the resilient member is located within an enclosed volume within the mobile core element.

4. An artificial intervertebral disc according to claim 3, wherein the enclosed volume is defined between the upper body and the lower body such that compression of the mobile core element reduces the free space within the enclosed volume, thereby containing the deformation of the resilient element.

5. (canceled)

6. (canceled)

7. (canceled)

8. (canceled)

9. An artificial intervertebral disc according to claims 1, wherein the first body includes a recess for receiving the resilient element.

10. An artificial intervertebral disc according to claim 9, wherein the recess is in the form of an elliptical bore.

11. An artificial intervertebral disc according to claim 10, wherein the elliptical bore defines a substantially flat, bottom surface and a substantially flat, perimetrical surface, in which the resilient element is, in use, received such that the perimetrical surface contacts the radial outer portion of the resilient element during compression of the mobile core element.

12. An artificial intervertebral disc according to claim 9, wherein the second body includes a protrusion carrying a surface on which the resilient element settles.

13. An artificial intervertebral disc according to claim 12, wherein the protrusion is received in the recess of the first body when the bodies are moved into their compressed configuration.

14. An artificial intervertebral disc according to claim 13, wherein recess and protrusion carry bearing surfaces which slide over each other as the mobile core element is compressed, thereby allowing lateral load transmission between the first and second bodies.

15. An artificial intervertebral disc according to claim 12, wherein the protrusion is elliptical.

16. An artificial intervertebral disc according to claim 15, wherein the protrusion carries a surface on which the resilient element settles.

17. An artificial intervertebral disc according to claim 15, wherein the elliptical protrusion is received in the elliptical bore when the first and second bodies are moved into their compressed configuration.

18. An artificial intervertebral disc according to claim 1, wherein the resilient element is an elliptical elastic component.

19. An artificial intervertebral disc according to claim 18, wherein the resilient element is a silicone insert.

20. An artificial intervertebral disc according to claim 1, wherein the resilient core element has means for locating the resilient element in the bore of the upper body.

21. An artificial intervertebral disc according to claim 20, wherein the resilient element is shaped complementary to the bore such that the resilient element locates automatically within the bore when received in the bore.

22. A method of approximating the natural behaviour of an intervertebral disc in an artificial intervertebral disc by compressing a compressible mobile core of the artificial intervertebral disc while restricting the deformation of a resilient element located in the mobile core, thereby obtaining an exponential increase in spring stiffness during compression.

23. A method according to claim 22, including restricting compression of the resilient element by locating the resilient element in an enclosed space.

24. A method according to claim 23, including reducing the volume of the enclosed space during compression of the mobile core.

25. A method according to claim 24, including allowing the resilient core to expand radially while compressing the resilient core axially.

26. A method according to claim 22, including locating the resilient core inside an elliptical bore inside an upper body of the core.

27. A method according to claim 26, including compressing the resilient core by moving a lower body relative to the upper body.

28. A method according to claim 27, including carrying the resilient element on the lower body and moving the lower body in the bore located in the upper body when compressing the resilient element.

29. A method according to claim 28, including sliding the lower body along a bearing surface of the bore inside the upper body when compressing the resilient insert.

30. A method according to claim 22, including preventing rotational movement of the resilient element relative to the upper body.
